(19) United States
(12) Patent Application Publication
(10) Pub. No.: US 2020/0200725 A1
(43) Pub. Date: Jun. 25, 2020

(54) SYSTEM AND METHOD FOR MONITORING CONDITIONS OF ORGANIC PRODUCTS

(71) Applicant: SUSTAINIO LTD., Herzeliya (IL)

(72) Inventors: Zohar KEREM, Rehovot (IL); Adi NAALI, Kfar Vitkin (IL); David KOREN, Hofit (IL)

(21) Appl. No.: 16/642,689

(22) PCT Filed: Aug. 29, 2018
(86) PCT No.: PCT/IL2018/050953

§ 371 Date: Feb. 27, 2020

(30) Foreign Application Priority Data
Aug. 31, 2017 (IL) 254235

(51) Int. Cl. G01N 33/00; G06Q 10/08; G06Q 30/00; G06N 20/00; G06F 16/21

(52) U.S. Cl. CPC G01N 33/0098; G06Q 10/087; G06F 16/21; G06N 20/00; G06Q 30/0185

(57) ABSTRACT

A monitoring system and method are presented for use in monitoring status of organic products. The system comprises a computer system comprising a data processing utility and being a part of and connected to a computer network. The data processing utility comprises and input interface, and a data analyzer. The input interface is configured and operable for receiving input data comprising a plurality of sensing signals independently received from a plurality of sensing systems via the computer network. The data analyzer comprises a product analyzer module configured and operable for extracting, from the sensing signals, one or more product-related signatures, and identifying from at least one of said one or more product-related signatures product type and status corresponding to said at least one product-related signature, and generating data indicative thereof, thereby enabling notifying a user with management data for managing use of said product.

### TECHNOLOGICAL FIELD

The present invention is generally in the field of smart sensing, in particular food industry or chain of supply of products, and relates to a system and method for monitoring organic products' conditions, such as freshness. The invention is particular useful for determining/monitoring freshness levels of foods.

### BACKGROUND

Various product types, and especially organic products, such as food products, deteriorate, degrade or undergo decomposition with time and with changes in surrounding environmental conditions, such as temperature and humidity. Monitoring of freshness level of such products may provide high-value data for storing, shipping and using such products. Suitable monitoring of product freshness or degrading status may be used for reducing costs associated with product waste, as well as health issues that may be associated with the good use of products such as food product in a fresh state or inadequate use in a deteriorated state. Also, from an end user's point of view, such monitoring of the freshness level of food products enables proper management and healthier consumption of the use of the food products being stored at home.

Today, the only indication of the food product's status is that provided by time-temperature indicators and resulting marking of the date of product expiration for use on the product (typically on the package). However, it has already been investigated that in many cases such date does not correspond to the real date of expiration, and as a result many products are thrown away before the actual expiration date. Indeed, such date actually reflects the time and temperature conditions to which the product has been exposed in between various stages during the product shelf-life/storage from manufacturer to market, and is based on statistical data about the product's shelf life under said conditions. Moreover, there is no data as to non-packed or open-packages products and as to home prepared or cocked foods. These may go faster deterioration, and may be kept in conditions that either favor a longer shelf-life at home or speed their deterioration and potential contamination.

Various techniques have been developed for monitoring the status of food products. For example, the Chinese patent publication CN105444513 describes an intelligent refrigerator capable of communicating with mobile phone. According to this technique, the intelligent refrigerator comprises a fresh-keeping box, a user terminal, a two-dimension code label, a control device, and a management module built on the user terminal. The two-dimension code label is used for storing the serial number of the fresh-keeping box. A two-dimension code recognition module obtains the serial number of the fresh-keeping box and stores the serial number in the management module, and then a camera module obtains a food image and stores the food image under the serial number of the fresh-keeping box. A box body humidity sensor detects the initial humidity value near food when the food is just put into the fresh-keeping box, continuously detects the real-time humidity near the food and sends a first humidity value to the control device. When the difference between the first humidity value and the initial humidity value exceeds a first humidity set value, the control device sends a first prompt message including the serial number of the fresh-keeping box to the management module, and the management module recognizes the serial number of the fresh-keeping box and displays the first prompt message under the food image. The intelligent refrigerator capable of communicating with the mobile phone realizes automatic monitoring and automatic prompt for the freshness degree of the food.

Another Chinese patent publication CN104020744 describes a fresh food sensor based on internet of things and cold insulation supply chain monitoring method. The method is used for monitoring the whole process of logistics of fresh food to avoid chain scission. The monitoring method comprises the steps that the fresh food sensor is started to read the RFID label data of a monitored object for RFID identification and is bound with the monitored object, and a module number corresponding to the RFID label is set to enable the monitored object, the fresh food sensor and a monitoring center to be driven synchronously in an integrated mode. Cold chain data are acquired, and primary processing is conducted on the cold chain data. Environmental data are compared with a temperature and humidity monitoring alarm critical value, and if the environment data are within the range of the set temperature and humidity monitoring alarm critical value, the shelf life and quality of the monitored object are calculated through an on-site data processor; obtained data are transmitted to a fresh food database of the monitoring center; data analysis and processing are conducted by a supply chain background server; network publicity is conducted; dispatching is conducted. According to the fresh food sensor based on the Internet of things and the cold insulation supply chain monitoring method, whole-course fresh food sensing, tracking and monitoring are achieved, and seamless linkage between the cold chain and the supply chain is realized.

### General Description

As described above, there is a need for determining a true status (quality, e.g. freshness) of various organic products, in particular food products. Today, users (e.g. individuals, enterprises) have no access to food's true freshness status and quality; this information is limited for large analytical labs only.

The present invention provides a novel approach for monitoring organic products' conditions by monitoring (detecting and analyzing) volatile organic compounds, which are produced during the deterioration and degradation of the organic products, and translating the detected data to the true status of the products indicative of their quality, in particular composition and freshness (safety) status. The technique of the invention enables continuous monitoring of such conditions and providing data about the real status of the product, and preferably also providing recommendations how this status, which is indicative of the (food) product quality, can be further taken into account for proper use of the product. Considering food products, the determination of the real status of a product should preferably be independent from any time-temperature indication labeled on the product.

In the description below, organic products being monitored by the technique of the present invention are referred to/exemplified as food products, and condition(s) being monitored is/are exemplified as freshness conditions. It should, however, be understood that the principles of the present invention are not limited to this specific type of products, and can generally be used for monitoring various conditions of organic products, other than food products, where these organic products are of the type that can be detected by sensing volatile organic compounds produced during the deterioration and degradation of the organic products. Also, it should be noted that the terms “freshness status” used herein should be interpreted broadly covering one or more conditions defining the quality (composition/safety) of the product, and generally any one or more conditions of a product characterizing its status which defines further use of the product.

The invention is based on the inventors' understanding that practically every organic product type and a change in its quality level can be identified by detecting/sensing a decomposition profile/pattern of the product. In case of food products, there are two mechanisms affecting the freshness/quality of a food product, associated with health factors and decomposition factors.

In this connection, reference is made to FIG. 1 graphically illustrating that, upon harvesting a food product, healthy related parameters characterizing the product, such as vitamins, anti-oxidants, characteristics odors, decrease (graph G₁), while decomposition related parameters of the product, such as protein, lipids, bacteria, acid, start to increase after harvesting (graph G₂), i.e. during the product storage, and can thus be monitored/sensed to describe the real status of the product and whether and how it can be used.

Thus, the invention provides a novel approach for characterizing the organic product by monitoring the product's odor/smell, i.e. volatile organic compounds produced during the normal activity, deterioration and degradation of the product. In other words, the invention provides a novel olfactory technique which provides information of the product's status, and preferably also instructions/recommendations as to whether and how to use the product in accordance with its current status. To this end, a system of the invention is configured for monitoring and analyzing a decomposition profile/pattern as being sensed in the vicinity of a product, and determining the product status.

As for the sensing technique it is based on the principles of “electronic nose” configured for detection of one or more parameters of molecules of predetermined types, as will be described further below. According to the present invention, a decomposition profile/pattern of a product, corresponding to the product current status, is defined by a unique, product-status related multi-parameter function of a number and a flow rate for each molecule from a predetermined set of molecules, as a function of time and environmental condition(s), e.g. temperature (i.e. dynamic change of the product status and accordingly the detectable decomposition profile/pattern of the product).

The inventors have shown that the sensing technique of the invention enables effective (high-quality, short time) determination of the food product “age” (status), e.g., eggs, fruits, fish, meat, etc. The sensing technique has two main stages: (1) identification of the (food) product type (i.e. class/group, such as meat vs fruit; and the type within the class/group such as banana vs mango); and (2) determination of the age of the identified product.

In some embodiments, the identification stage is based on statistics and data, being created and updated by self-learning technique, and the type determination stage is based on specific data processing (software product/algorithmic) which is in turn continuously optimized by the self-learning technique. In other words, the data processing utility may be an expert system preprogrammed for performing the complete sensing technique. As will be described further below, the sensing technique involves physical sensor(s) which is/are in data communication with a control unit, which may be a server computer system, preferably implementing cloud computing technique.

According to its one aspect, the present invention provides a monitoring system for use in monitoring status of organic products, comprising a computer system having a data processing utility and a non-transitory computer readable memory and being a part of and connected to a computer network. The data processing utility comprises: an input interface configured and operable for receiving input data comprising a plurality of sensing signals independently received from a plurality of sensing systems via the computer network; and a data analyzer comprising a product analyzer module configured and operable for extracting, from the sensing signals, one or more product-related signatures, and identifying from at least one of said one or more product-related signatures a product type and a status corresponding to said at least one product-related signature, and generating data indicative thereof, thereby enabling notifying a user with management data for managing use of said product.

It should be understood that such a “computer system” is constituted by a software product installed on a “server” system, e.g., “cloud computer”, or may be “client computer”, e.g. API in client's personal communication device, such as a mobile phone, or various modules of the computer system may be distributed between the server and client computers (i.e. distributed software).

It should thus be understood that the term “control system” should be interpreted broadly, covering local controllers (data analyzers) in data communication with the sensing unit/system, as well as cloud computing based system. The latter is a type of Internet-based computing that provides shared computer processing resources and data (such as servers, storage and applications) to computers and other devices through the computer network (or communication network), such as the Internet. Cloud computing and storage solutions provide users and enterprises with various capabilities to store and process their data in either privately owned, or third-party data centers that may be located far from the user-ranging in distance from across a city to across the world. Thus, the invention provides for using cloud computing technique, according to which a central data analyzer (software) is used to receive the sensing data from multiple products' storage locations and using these multiple data sources for optimizing the above-mentioned identification of the product types and product status monitoring (e.g. utilizing self-learning modes, models' optimization, etc.).

It should also be noted that, for the purposes of the present application, the term “user” should be interpreted broadly, covering any “client side”, being an end user at home, storage entity, etc.

Further, it should be noted that the terms “sensing unit” or “sensing element” or “sensor device”, as used herein, refer to a sensor (single-element or multi-element sensor) implementing any known suitable sensing technique(s) for sensing/detecting chemical and/or biochemical and/or physical parameters characterizing media in the vicinity of the sensor (as well as dynamic changes of such parameters) and translating them into “readable” signals (e.g. electrical signals). Chemical and biochemical sensing provide for determining material-related parameter(s) of the media (concentration/amount of chemical and/or biological quantity); while physical sensing provides for determining parameter(s) indicative of physical interaction with various materials, as well as such parameters as pressure, displacement, temperature within the media. As describe above, the sensing data to be analyzed is indicative of number and flow rate for each molecule from a predetermined set of molecules, as a function of time and environmental condition, and possibly also the type of a sensor (sensing technique) being used.

The monitoring system may further include a manager utility configured and operable for analyzing the data indicative of the product type and status, and generating notification data for managing use of said product.

The monitoring system may further comprise a communication interface utility configured and operable for data communication with a user's communication device via said computer network for communicating said notification data to the user's communication device.

In some embodiments, the data processing utility is configured and operable to access and manage a database for storing data about various types of products, where each product type is associated with a respective unique set of product decomposition patterns.

The data processing utility may be configured and operable to manage said database for storing each product decomposition pattern with associated sensing data for sensing product decomposition pattern.

In some embodiments, the sensing data comprises data indicative of characteristics of one or more sensing systems from which said sensing signals are originated. In some embodiments, the sensing data comprises data indicative of one or more environmental conditions to which the sensing systems, producing said sensing signals, are exposed.

In some embodiments, wherein said product analyzer module is configured and operable for identifying the sensing data in the sensing signals being received from the sensing system.

In some embodiments, product analyzer module is configured and operable for identifying the product type and status by applying model-based analysis to the extracted product-related signature using at least one selected model data comprising multi-parameter functions describing product decomposition patterns, said model-based analysis comprising a data fitting procedure between the extracted product-related signature and the selected model data.

In some embodiments, the multi-parameter function is indicative of a sensing signal as a time function of at least one parameter for each molecule from a predetermined set of molecules, being sensed over sensing time.

In some embodiments, the at least one parameter of the molecule comprises either one or both of a number of the molecules of a certain type and a flow rate of said molecules being sensed over the sensing time.

In some embodiments, the multi-parameter function is further indicative of the sensing signal as a function of one or more environmental conditions to which the sensing system is exposed during said sensing time.

In some embodiments, the one or more environmental conditions comprise at least one of temperature and humidity conditions.

In some embodiments, the data analyzer comprises a learning module configured and operable for analyzing the product-related signatures in the independently received sensing signals relating to the same product types, and optimizing database for storing model data including various models describing product decomposition patterns characterizing various products.

In some embodiments, the data analyzer further comprises a verification module configured and operable for verifying the product type by analyzing the product-related signature extracted from the sensing signal over one or more other product-related signatures in the received sensing signals.

In some embodiments, the data processor is configured and operable to manage the database for storing said data about various types of products by creating and storing in said database reference data comprising measured sensing signals from a plurality of test samples of known product types as functions of time and one or more environmental conditions to which a sensing system is exposed during collection of said measured sensing signals, for various types of the sensing systems and sensing modes.

The invention further provides a storage system being a part of and connectable to a communication network, the storage system comprising a database comprising data indicative of sensing signals from a plurality of samples of known organic product types and various quality level (e.g. freshness) statuses for each product type, each sensing signal being a function of time and one or more environmental conditions to which a sensing system is exposed during collection of said sensing signals, for various types of the sensing systems and sensing modes.

In some embodiments, the storage system further comprising, for each product type and status, data indicative of whether and how said product with said status can be used.

The invention further provides a storage system being a part of and connectable to a communication network, the storage system comprising a database comprising notification data to be provided to users of multiple organic products, said notification data comprising, for each of the multiple organic products and each of its different freshness statuses, data indicative of whether and how said organic product with the specific freshness/quality/safety status can be used.

The invention further provides a method of creating a database for use in evaluating a product status, the method performed by a computer system comprising a processor and a non-transitory computer readable memory and being a part of and connected to a computer network, the method comprising:

(i) independently receiving and storing, in said non-transitory computer readable memory, input data comprising a plurality of measured signals from multiple sensing systems via the computer network, said plurality of the measured signals comprising sensing signals measured by one or more of the sensing systems from different products of the known type and different statuses for each of said products over different sensing time intervals and different environmental conditions of said one or more of the sensing systems during the sensing times; and

(ii) analyzing the input data to assign, to each type of the known product, a set of the sensing signals corresponding to product decomposition profiles over time, as functions of the environmental conditions, product status and sensing modes;

(iii) creating the database in which the product decomposition profiles are stored together with the corresponding assigned product types; and

(iv) enabling access to said database via the computer network.

Further provided is a personal communication device configured to be a part of and connected to a communication network, the device comprising a non-transitory computer readable memory storing an application program interface comprising a manger utility configured and operable to be responsive to input data indicative of product-related data comprising type and quality status of a certain organic product, for analyzing said product-related data, and generating output data comprising notification data describing whether and how said product with said status can be used.

According to another aspect, there is provided a sensing system comprising: a sensing unit comprising one or more sensors configured and operable in predetermined one or more sensing modes for continuously detecting various molecules, and generating sensing data comprising data indicative of detected molecules over time and data indicative of the sensing mode used for the detection of said molecules over time; and a communication utility for wireless communication of the sensing data to a remote monitoring system.

According to another broad aspect of the invention, it provides a storage system being a part of and connectable to a communication network, comprising a novel database which stores data indicative of sensing signals from a plurality of samples of known organic product types and different quality statuses for each of the product types, such that each sensing signal corresponds to a function of time and one or more environmental conditions to which a sensing system is exposed during collection of said sensing signal, for various types of the sensing systems and sensing modes.

Also, the database may further comprise, for each product type and status, data indicative of whether and how said product with said status can be used.

The invention also provides a method of creating the above database comprising:

(i) independently receiving and storing, in said non-transitory computer readable memory, input data comprising a plurality of measured signals from multiple sensing systems via the computer network, said plurality of the measured signals comprising sensing signals measured by one or more of the sensing systems from different products of the known type and different statuses for each of said products over different sensing time intervals and different environmental conditions of said one or more of the sensing systems during the sensing times; and

(ii) analyzing the input data to assign, to each type of the known product, a set of the sensing signals corresponding to product decomposition profiles over time, as functions of the environmental conditions, product status and sensing modes;

(iii) creating the database in which the product decomposition profiles are stored together with the corresponding assigned product types; and

(iv) enabling access to said database via the computer network.

In a further broad aspect, the invention provides a personal communication device configured to be a part of and connected to a communication network, the device comprising a non-transitory computer readable memory storing an application program interface comprising a manger utility configured and operable to be responsive to input data indicative of product-related data comprising type and quality status of a certain organic product, for analyzing said product-related data, and generating output data comprising notification data describing whether and how said product with said status can be used.

According to yet another broad aspect of the invention, it provides a storage system being a part of and connectable to a communication network, comprising a novel database comprising notification data to be provided to users of multiple organic products, said notification data comprising, for each of the multiple organic products and each of its different quality statuses, data indicative of whether an how said organic product with the specific freshness status can be used.

Further provide by the invention, is a sensing system comprising: a sensing unit comprising one or more sensors configured and operable in predetermined one or more sensing modes for continuously detecting various molecules, and generating sensing data comprising data indicative of detected molecules over time and data indicative of the sensing mode used for the detection of said molecules over time; and a communication utility for wireless communication of the sensing data to a remote monitoring system.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 graphically illustrates a dynamic change in healthy-related and decomposition related parameters of a food product after harvesting, and how this data can be used for determining the real (true) status of the product and whether and how it can be used at successive stages over time;

FIG. 2A is a block diagram of a technique of the invention for monitoring status (typically freshness/quality) of organic products (e.g. food products) located at various storage locations;

FIG. 2B is a diagram of the operational principles of the technique of the invention for communicating and processing sensing data from multiple sensing systems using cloud computing technique;

FIGS. 3 and 4 are block diagrams exemplifying functional modules of a control system (monitoring system) of the present invention;

FIG. 5 is a flow diagram of a method of the invention implemented by the control system of the invention for creation and use of a database for storing data about various types of organic products at different freshness statuses and corresponding decomposition profiles (signatures) detectable over time under different environmental conditions using different sensing modes;

FIGS. 6A to 6D show some results for sensing volatile emission of molecules [Mira, S. et al., 2016, Volatile emission in dry seeds as a way to probe chemical reactions during initial asymptomatic deterioration. Journal of experimental botany, 67(6), 1783-1793.), wherein: FIG. 6A illustrates various examples of sensing signals for volatile emission of different types of molecules which are characteristic of various paths of deterioration and decomposition of food products; FIGS. 6B-6D illustrate of the same for three types of seeds, Lactuca sativa (Asteraceae), Eruca vesicaria (Brassicaceae) and Carum carvi (Apiaceae) under varying environmental conditions (humidity conditions), demonstrating the potential to determine the botanical source of closely related and dry seeds by sensing of volatiles. These data also exemplify how the sensed signals provide for distinguishing between different food product classes/groups, as well as the feasibility of the sensing sessions; and

FIG. 7 illustrates experimental results obtained by the inventors for the technique of the present invention, demonstrating the dynamics of the degradation and decomposition of fruits, in this case using a combination MEMS-MOS sensors, demonstrating the capability of sensors to translate data on volatiles to indicative output on the change in the product status.

### DETAILED DESCRIPTION OF EMBODIMENTS

The present invention provides a novel technique for monitoring/determining the true status (and quality) of an organic product, such as a food product and the dynamic changes in such status with time and environmental conditions, and also provides for notifying a user about such status and preferably also providing user with the recommendations as to how to use the product. As indicated above, the present invention is particularly useful for monitoring food products, and is therefore described below with respect to this specific application, which is an example not limiting the general principles of the invention.

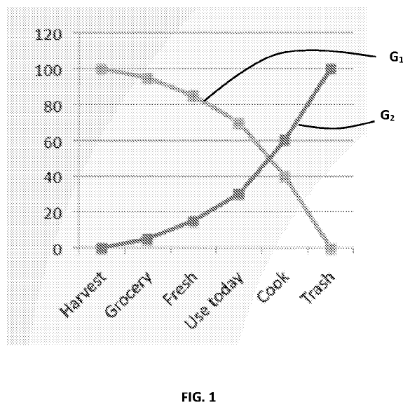

As also described above, and specifically referred to FIG. 1, the invention is based on the inventors' understanding of the mechanisms and factors/parameters describing a dynamic change in the freshness status of a food product.

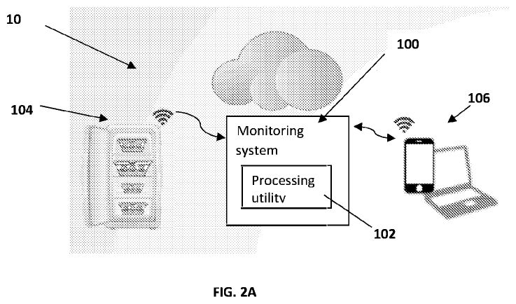

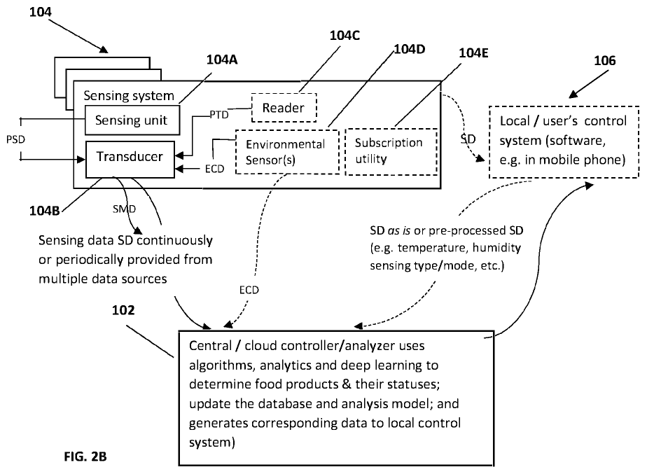

Referring to FIGS. 2A and 2B, the principles of the technique of the invention are schematically illustrated. FIG. 2A is a block diagram of a system configured to implement the invention, by data communication between remote devices/systems involved in the implementation of the invention via a communication network 10. In the present not limiting example such communication is implemented based on the principles of cloud computing techniques.

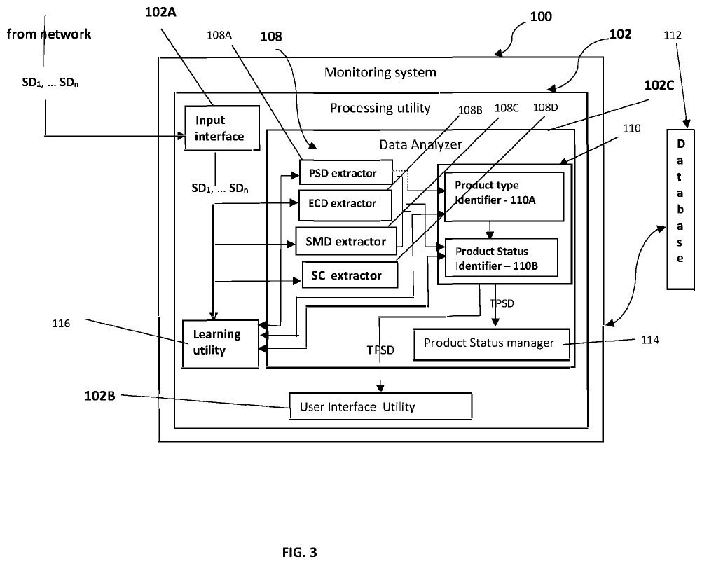

The system 10 includes a monitoring system 100 which includes or is configured as a computer system configured and operable according to the present invention. The computer system 100 is a part of and connected to the communication network 10, and includes inter alia a data processing utility 102 (software product). The data processing utility 102 is configured according to the invention for communication with multiple data sources, generally at 104, at various remote locations to independently receive sensing signals SD from the multiple data sources and process these multiple sensing signals; and for communication with user's personal communication devices, generally designated 106, to provide output data indicative of the processing results. The configuration and operation of the data processing utility 102 will be described more specifically further below with reference to FIG. 3.

For the purposes of the present application, the data source 104 is a sensing system including a sensing unit 104A which provides (continuously or periodically) sensing signals SD, and a communication utility (transducer) 104A for communication (via wires or wireless using any known suitable signal/data communication techniques) of data indicative the sensing signals SD to remote receiver(s). The sensing unit 104A is located in the proximity of the organic products which deteriorate with time and are thus to be monitored (e.g. the sensing unit is located within a storage space containing various food products), such that the sensing unit (or at least sensing region(s) thereof) is exposed to the environmental conditions (temperature, humidity, etc.) to which the products are exposed.

The sensing unit 104A includes one or more sensors (chemical and/or biochemical and/or physical sensor(s)) of any known suitable type(s) having sensing region(s) configured for interacting with different molecules of predetermined types characterizing materials being decomposed by the various products in said space, and generating sensing signals. The sensing unit may include a plurality of sensors, or a so-called “sensor matrix”. The sensing signals produced by the sensing unit 104A describe said interaction by one or more predetermined parameters and are therefore indicative of the product status data PSD, as will be described more specifically further below.

As also shown in the figure, in some embodiments, the sensing system 104 may include a reader unit 104C capable of reading data provided on the product and comprising information about the product type (product type data PTD), as the case may be. Such information may for example be embedded/encoded in a product identification code (e.g. barcode) that might be carried by one or more products in the storage space, and being indicative of the product type. As also shown in the figure, the sensing system 104 may include an environmental sensing unit (one or more environmental sensors) for sensing environmental condition(s) by monitoring such parameter(s) as humidity and/or temperature in the surrounding space, and producing environment condition data ECD.

Further, it should be understood that the sensing system 104 (i.e. user owing the sensing system) is typically a subscriber of the monitoring system 100, and is assigned with a subscription code. Accordingly, either the sensing system 104 or the respective personal communication device(s) 106 is installed with an appropriate subscription utility 104E which operates to accompany the sensing signals, being output from the sensing system, with data indicative of the subscription code. It should be understood, although not specifically shown, that alternatively or additionally, such subscription utility 104E may be part of the communication utility 104B.

Thus, sensing signals/data SD produced by the sensing system include the smell-related signals (produced by the sensing unit 104A) indicative of the product-related data, and preferably also include data indicative of environmental condition(s) produced by environmental sensing unit 104D, and may or may not further include the product type data (provided by reader 104C. It should be understood, and is shown in the figure by dashed lines, that environmental sensing unit 104D (or at least one sensor thereof) may be part of the sensing system 104, or the sensing system 104 may be configured to communicate with such one or more environmental sensors separately installed in the storage space. The environmental condition data ECD may thus be transmitted to the central controller 102 by communication utility 104B, or may be directly transmitted to the central controller 102 by the external environmental sensor(s) as the case may be.

The communication utility 104B is configured and operable according to any known suitable technique for properly formatting and transmitting the sensing signals, using any known suitable communication (be it wireless or wired)/protocol to a remote data analyzer. It should be noted that the communication utility 104B may be preprogrammed to assign to the product-related/product-status data PSD being sensed by the sensing unit 104A, data indicative of the sensing mode SMD (e.g. type of sensor being used in the sensing unit 104A). Thus, the output data provided at the data source side, which is referred to herein as “sensing data” or “sensing signals” SD, actually includes at least the product-status data PSD as being sensed from various products in the storage space (carrying information about products' decomposition profiles), and may further include one or more of the environmental condition data ECD and the sensing mode related data SMD, and in some cases may also include product type data PTD.

As also exemplified in the figure, the entire sensing data/signals SD or at least some of its portions, may be directly communicated to the central controller 102, or via the personal communication device 106. For example, as shown in the figure, data portions including the product-status data PSD (and possibly also the product type data PTD) and/or the environmental condition data ECD and/or the sensing mode data SMD produced by the different sensors 104A, 104C and 104 are independently communicated to the personal communication device 106, where all these data portions are combined into the final output data SD, which is transmitted by the device 106 to the central controller 102 via the network 10.

Reference is now made to FIG. 3 showing schematically, by way of a block diagram, some of the functional modules of the controller 100 (monitoring system) configured and operable according to the present invention. As described above, the system 100 is a computer system comprising a data processing utility 102 and being a part of and connected to the computer network. The processing utility 102 includes an input interface module 102A configured for data communication, via the network, with multiple data sources providing sensing data/signals. It should be understood that the sensing data or at least part thereof may be provided directly from sensing systems and/or from personal communication devices. In the specific not limiting example of FIG. 3, the processing utility 102 also includes user application interface 102B which is configured for data communication, via network, with multiple personal communication devices to provide output data indicative of the products' statuses.

Also provided in the processing utility 102 is a data analyzer module 102C for processing the received sensing signals and generate the output data. Thus, input interface module 102A receives input data from the network comprising a plurality of sensing data pieces SD₁, . . . SDₙ independently provided from each of a plurality of n sensing systems 104. It should be understood that each sensing data piece SDᵢ includes multiple sensing signals associated with various products to which the sensing unit of the respective sensing system is exposed, and generally the sensing signals are not assigned with/carry the respective product type data. The processing utility 102 is capable of identifying the product type data in the received sensing data piece.

More specifically, the data analyzer module 102C includes a data type extractor utility 108 having a product signature extractor module 108A which is preprogrammed (configured and operable) to extract, from the sensing data pieces SD₁, . . . SDₙ, one or more product-related signals/signatures PSD (bit stream); environmental condition data extractor 108B, and possibly also sensing mode data extractor 108C. The product related signatures are then processed/analyzed by a product identifier utility 110, which is preprogrammed (configured and operable) to analyze the product related signatures using reference data stored in a database 112 (accessible via the network) and identifying, from the product related signatures, the corresponding products' statuses. As exemplified in the figure, product identifier utility 110 includes a product type identifier module 110A which processes the extracted signatures and identifies the product type, and a product status identifier module 110B which analyzes the respective signature for the identified type of product and the environmental condition(s) being sensed using the identified sensing mode, and determines the true/real status of the product (e.g. freshness level), and generates output data being true product status data TPSD. As an example, if the system identifies a signature of a chicken, it provides a signature related data to the status identifier, which verifies the chicken freshness level, and the outcome in-turn is populated to the end device (user device, e.g., mobile phone) for status update. The user can thus see a visual indicator that a chicken with said status has been identified in his sensed area.

The so-determined true product status data TPSD may be directly transmitted to the user side via user application interface 102B. To this end, the processor utility 102 further includes a user identifier module 102D capable of identifying, in the sensing signals, the respective subscription code SC and determining network location of the respective user's communication device.

It should be noted that the function of sensing mode data extractor 108C may be performed by the user identifier module 102D. For example, data indicative of the sensing mode may be part of the user's subscription data.

Preferably, the true product status data TPSD is further analyzed by a product state manager module 114 capable of determining the so-called “product use data” PUD for the product with said status, and generate notification message to the user. To this end, the product state manager module 112 accesses respective reference data previously determined and stored in the database. The reference data is a so-called theoretical or modeled data previously created and stored, and the analysis of the sensing signals is performed using a fitting procedure between the sensed/measured data and the selected modeled data.

As shown in the specific not limiting example of FIG. 3, the product state manager module 110 may be part of the processing utility 102 at the monitoring system. Alternatively, the configuration may be such that the product state manager module 110 is a part of the application (software) installed in a personal communication device (106 in FIGS. 2A-2B).

The data processor utility 102 is configured as an expert system having a learning utility 116 configured and operable for performing a self-learning mode for updating/improving the performance of the data analyzer module 102C for the extraction of product-related signatures PSD from the received sensing signals and determination of the product types and statuses. To this end, the learning utility receives and analyzes multiple, independently provided sensing signals and analyses them one over the others to verify the signatures characterizing the specific product type under different environmental conditions and different sensing modes, and update the database.

The database 112 is created using the technique of the invention by applying multiple measurements to products of various known types under controllably varying environmental conditions, and preferably also using different known sensing modes (e.g. various types of sensors). The database 112 thus sores data about various types of products, where each product type is associated with a respective unique set of sensing signals/signatures characterizing product decomposition patterns over time under different environmental conditions. The creation and use of such database will be described more specifically further below.

As indicated above, the sensing signals/data generated by the sensing unit (e.g. sensor matrix) includes data indicative of product-related signatures corresponding to environmental condition(s) during sensing/measurement time to which the sensing unit is exposed during this time. In this connection, it should be understood that different food products and types of food products can be distinguished by such factors as heat dose, cold dose, and humidity dose.

Heat dose is determined by the product's exposure time to temperature conditions above 20° C. Cold dose is determined by the product's exposure time to temperature below certain temperature, e.g. 10° C. (typical refrigeration temperature). Humidity dose is determined as time during which the product under predetermined humidity conditions, for example relatively low humidity condition corresponds to humidity below 30%, medium humidity condition corresponds to humidity between 30% and 70%, and high humidity condition corresponds to humidity above 70%. The relatively low humidity prolongs the life time (i.e. fresh-state life time) of product, medium level humidity reduces such life time, but at a lesser extent than high humidity does.

The heat dose (heat units) defines an addition to chronological age of food product (types of food) and the cold dose (cold units) defines reduction from the chronological age, where addition and reduction vary between different food products (types of food). The heat units and cold units can thus be described as the functions of time t, Y(t)ₓ and Y(t)_{f}, where x and f are respective temperature coefficients for different types of foods.

The learning utility 116 is thus also configured and operable for performing learning algorithms to define the heat dose and cold dose for each food type, as well as effect of the humidity conditions on different food types. It should be understood that these factors are dependent on one another, e.g. for a given food product the heat dose and/or cold dose may be different for different humidity conditions.

As also described above, sensing signals/data may be formed by output of multiple sensors or sensing units. For example, considering measurements by three sensors, freshness status of food product may be described as the following function:

Freshness=a·S⁽¹⁾ₒᵤₜₚᵤₜ+b·S⁽²⁾ₒᵤₜₚᵤₜ+c·S⁽³⁾ₒᵤₜₚᵤₜ+cold·(−g·t)+heat(+1.5h·t)+Humidity(low)·(−1i·t)+Humidity(medium)·(0.1t·t)+humidity(high)·(+4i·t)

wherein: a·S⁽¹⁾ₒᵤₜₚᵤₜ is the output of sensor 1;

g is the respective cold dose/temperature coefficients for different types of foods;

h is the respective heat dose/temperature coefficients for different types of foods;

i is the respective humidity coefficients for different types of foods.

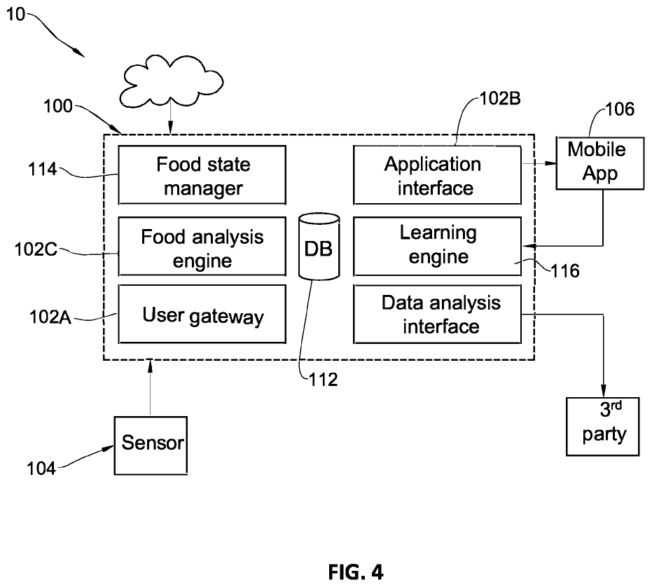

FIG. 4 shows in a self-explanatory manner a block diagram of the main functional modules/utilities (software and/or hardware) installed in the monitoring system 100 of the present invention for performing the above-described technique of the invention utilizing cloud computing for data processing and data communication via the network with the remote sides involved, i.e. sensing systems 104, user's communication devices 106 (e.g. mobile phones), database 112. Also as shown in the figure, relevant data can be communicated to any other user/authorized entity involved. Such a third party will thus be able to utilize the real time food data for his specific application, such a specific application which can be but not limited to an application that provides the health care entity the usage of food and dietary behavior with elderly people.

It should be understood that generally, the processing utility 102 of the invention may be entirely installed in a user's communication device and configured for accessing the remote database at the server via the network.

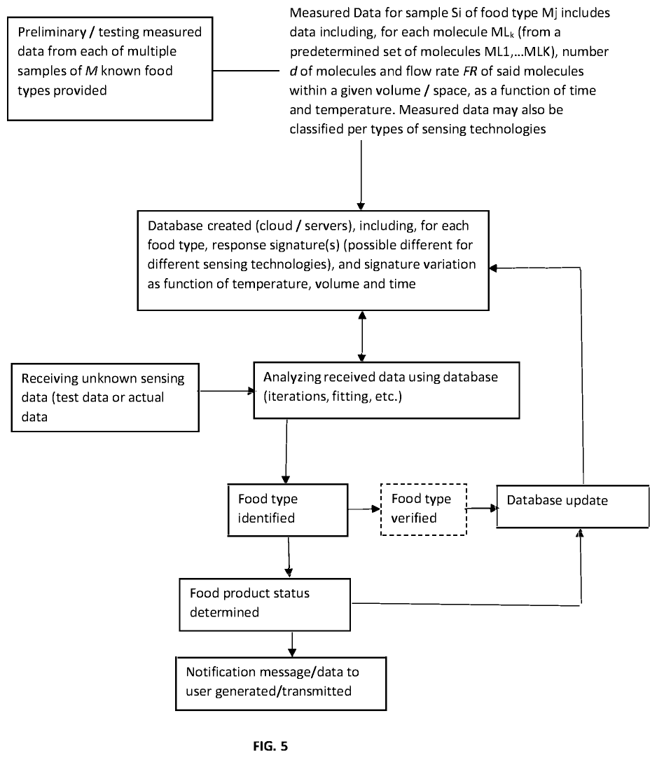

Reference is now made to FIG. 5 schematically illustrating a flow diagram of a method of the invention for creating and using the database configured as described above, namely database that sores data about various types of products, where each product type is associated with a respective unique set of sensing signals/signatures characterizing product decomposition patterns over time under different environmental conditions.

Initially, each of multiple products of N different types (e.g. chicken, various types of fish, eggs, etc.) is monitored/measured over time, under different environmental conditions (e.g. temperature and/or humidity), and preferably also using different sensing modes (types of sensors, the size of sensing volume/space, distance between the product and sensing surface). The sensors used in the measurement sessions are of the type which, when being located in the vicinity of the product and exposed to the same environmental conditions to which the product is exposed, is capable of interacting over time with molecules of a predetermined set of K types of volatile molecules ML₁, . . . ML_{K} (as exemplified below), and determining, for each molecule type, a number d of molecules and flow rate FR of said molecules within a given volume/space, as a function of time (and preferably also environmental condition(s) such as temperature and humidity). The measured data may also be classified per types of sensing technologies/modes. Thus, the measure data may be a multi-parameter function describing the decomposition profile of the product over time for given environmental condition(s).

The following Table exemplifies the volatile molecules detectable for the purposes of the present invention and indicative of the product decomposition profile.

| | Chemical families | Volatiles |
|---|---|---|
| lipids | lipid-derived | hept-(E)-enal |
| | carbonyls | Oct-(E)-enal |
| | | deca-2,4-di-(EE)-enal |
| | | heptan- 2-one |
| | | octan-2-one |
| | | 3-hydroxy-butan-2-one |
| | | octane-2,3-dione |
| | alcohols | heptanol |
| | | hexanol |
| | Volatile acids | acetic acid |
| Protein | Strecker | methyl propan-2-al |
| | aldehydes | Methyl butan-2-al |
| | | methyl butan-3-al |
| | | benzaldehyde |
| | Voatile and | diisopropylamine |
| | bigenic | 2-aminobutane |
| | amines | diisobutylamine |
| | | 1-aminopentane |
| | | dibutylamine |
| | | pyridine |
| | | decylamine |
| | | dicyclohexylamine |
| | | 2,6-dimethylaniline |
| Synthetics | fumigants | methyl bromide |
| | | phosphine |
| | | 1,2-dichloroethane |
| | Fragrances | Decanol |
| | | hydroxyisohexyl 3-cyclohexene |
| | | carboxaldehyde |
| | | sorbitan sesquioleate |
| | | Phenoxyethanol |
| | | Salicylaldehyde |
| | | Glutaraldehyde |
| | | hydroperoxides |
| | | trimethylbenzenepropanol |
| | | dipropylene glycol |
| | | benzyl benzoate |
| | | oil of turpentine |
| | | metals (nickel sulphate, cobalt chloride) |
| | | 2-hydroxy-5-octanoylbenzoic acid |
| | | capryloyl salicylic acid |
| | | b-lipohydroxy acid |
| | | methylchloroisothiazoline/methylisothiazoline |
| | | methyldibromoglutaronitrile |
| | | trimethylbenzenepropanol |
| Natural | monoterpenoids | 3-ethyl-3-methylheptene |
| volatiles | | alpha - and beta -pinene |
| from seeds | | beta -phellandrene |
| | | 4-methylheptane |
| | | 2,4-dimethylheptane |
| | | d-limonene |
| | | γ-decalactone |
| | | 3-carene |
| | aldehydes | pentanal |
| | | 2-octenal |
| | | heptanal and 2-heptenal |
| | | hexanal |
| | | 2-octenal |
| | | heptanal and 2-heptenal |
| Baterial | Amino acid | 2-methyl 1-Propanol |
| spoilage | catabolism | 3-methyl-Butanal |
| | | 2-methyl-Butanal |
| | | 3-methyl- 1-Butanol |
| | | 2-methyl- 1-Butanol |
| | | 2-methyl- 2-Butenal |
| | | dimethyl- Disulphide |
| | Residual | 2,3-Butanedione(diacetyl) |
| | glycogen | 2,3-Pentanedione |
| | catabolismd | 2-Butanone |
| | | 3-hydroxy- acetoine |
| | | 2,3-Butanediol |
| | Fatty acid | 2-Butanone |
| | catabolism | 1-Penten-3-ol |
| | | 2-Pentanone |
| | | 3-Penten-2-one |
| | | 2-Pentanol |
| | | 3-Hexanone |
| | | 2-Hexanone |
| | | Hexanal |
| | | 3-Hexanol |
| | | 2-Heptanone |
| | | Nonanal |
| | | Decanal |
| | Multiple | 2-Propanone |
| | origins | 1-Propanol |
| | | Acetic acid |
| | | Ethyl acetate |
| | | n-Propyl acetate |
| | | Ethyl butanoate |

The inventors have found that using a limited set of molecules enables determination/sensing of the decomposition profiles of multiple different types of food product, and analyzing dynamics of such molecule response of the product enables detection of true status of the product. More specifically, the inventors have found that the decomposition profile of a product (characterizing its true status) can be defined as follows:

$$\begin{matrix} f ( decay ) = Σ δ protein + Σ δ lipids + Σ δ bacteria + Σ δ acid & ( 1 ) \\ f ( deterioration ) = {lim}_{t \to ∞} ( ( δ volatiles characteristics + δ volatiles positive ) / t ) + {lim}_{t \to ∞} ( δ HPC / t ) & ( 2 ) \\ f ( fishdecay ) \neq f ( meatdecay ) & ( 3 ) \\ f ( fishdecay ( salmon ) ) ≅ f ( fishdecay ( sea bass ) ) & ( 4 )\end{matrix}$$

Food decay processes are divided into two distinct processes:

(1) Decay where degradation components are produced and accumulated. The accumulation of such compounds results in the formation of bad flavors and aromas, compounds with either potential or proven adverse effects on human health. These processes lead to the release of many volatile compounds to the surrounding atmosphere of every food product. The production and release of volatiles depends and relates to the chemical composition of the product, in terms of major chemical families, amongst which one can include proteins, lipids (oils and fats), lipid soluble compounds, carbohydrates which are composed of simple sugars and polysaccharides, reducing compounds which are also known as antioxidants, pigments, and organic salts. The production of the above volatiles is also a consequence of the composition of each of the above families, e.g. in the lipid family different products will be produced from the C18 omega 3 fatty acids e.g. linolenic acid then from the C20 omega 3 fatty acids, e.g. Eicosapentaenoic acid (EPA). Similarly, different products are expected from different amino acids in a protein, and the chemical reactions of proteins in the presence of different sugars will lead to the production of different products and more specifically a large and versatile array of volatiles. The formation of aroma-active strecker-aldehydes presents another sample, where specific volatiles result from specific raw materials. Specific food products will lead to the formation of different aldehydes, such as 3-methylbutanal (malty) or phenylacetaldehyde (honeylike) that are formed from the corresponding amino acids leucine and phenylalanine, respectively, when reacted in the presence of alpha-dicarbonyl compounds.

(2) Deterioration leading to reduction of positive and health promoting compounds from food. During the above described aging of food products and the production of compounds with negative effects, compounds that are required to maintain the normal physiological functions, and cannot be synthesized by human are degraded. Such compounds, called essential nutrients, must be obtained from a dietary source and are indispensable for all metabolic processes and the proper physiological functions of tissues and organs, and in certain developmental and pathological states. The production of volatile degradation products, that are sensed in this patent, is translated into reduction in the nutritional and health promoting values of every food product. For instance, in fruits, vegetables and salads, the age pf a food product can be directly translated into loss in vitamin C, which is then translated into freshness level of the said food product, reaching an end point where the food has lost all its vitamin C and if this was its last positive compound, the said food product might or should be trashed.

The above understanding leads to two base definitions in the food decay scheme based on Functions (3) and (4):

(3) provides a very intuitive condition (given the above and as shown in the figures) according to which fish decay function provides a significantly different value from the function of meat decay;

(4) is provided on the assumption that given a large enough sample base e.g. big database, a specific fish within the dataset, e.g., salmon, will provide a slightly different value from another type of fish decay, e.g., seabass.

Thus, multiple product-samples of different types undergo the above-described test measurements, the sensing signals are analyzed to assign, to each type of the known product, a set of the sensing signals corresponding to the product decomposition profiles over time, as functions of the environmental conditions, product status and sensing modes.

Such sensing signals are stored in the database together with the associated product type. These sensing signals present “modeled data”, which is then used for processing and analyzing various unknown sensing data independently received from multiple sensing system via the network, as described above, using iteration and fitting procedures.

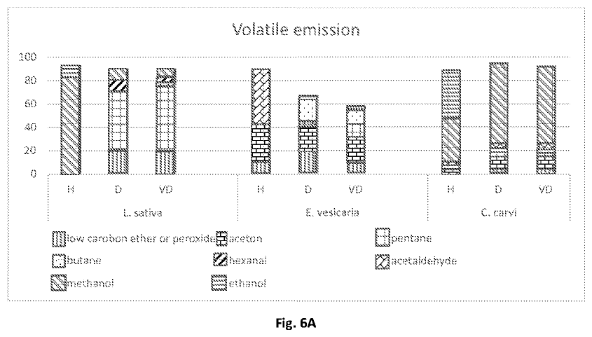

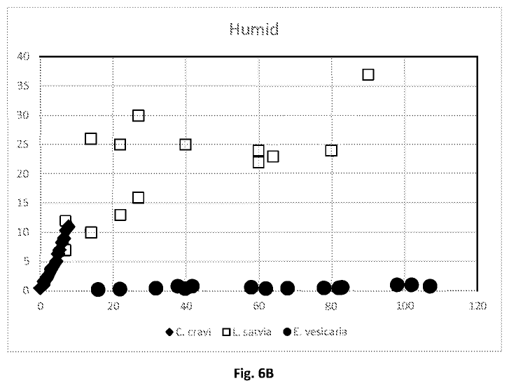

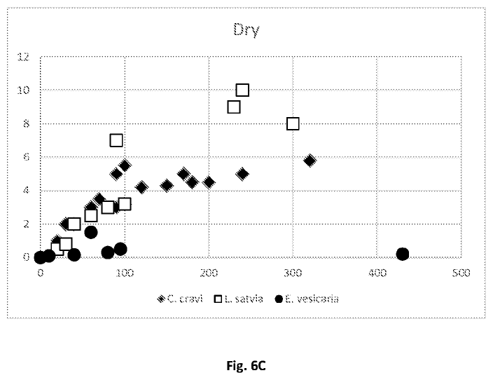

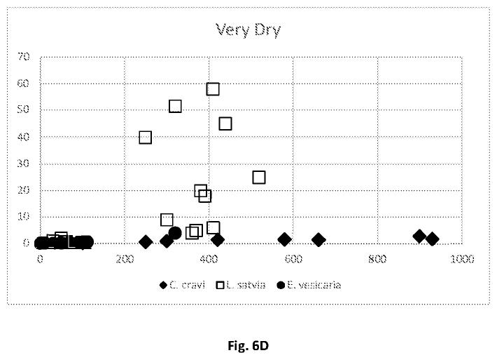

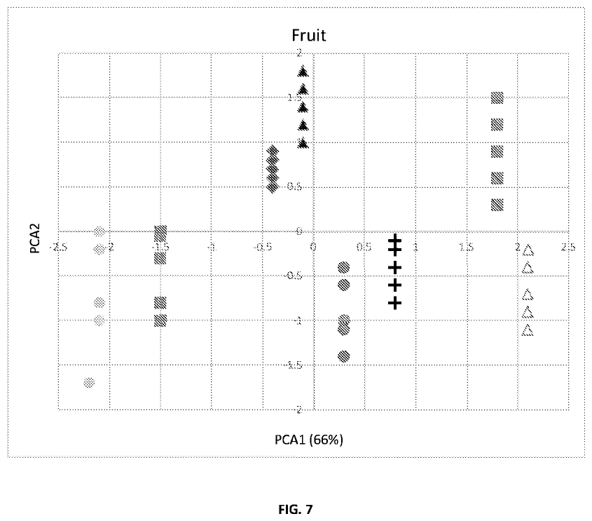

Reference is made to FIGS. 6A-6D and FIG. 7 showing the sensing volatile emission of molecules [Mira, S. et al., 2016, Volatile emission in dry seeds as a way to probe chemical reactions during initial asymptomatic deterioration. Journal of experimental botany, 67(6), 1783-1793.)—FIGS. 6A-6D; and the experimental results obtained by the inventors demonstrating the dynamics of the degradation and decomposition of fruits—FIG. 7.

FIG. 6A illustrates various examples of sensing signals for volatile emission of different types of molecules which are formed in various seeds during deterioration and are characteristic of decomposition of food products. More specifically, the figure shows the evolution profile of emission of volatile molecules as a result of decomposition of three types of seeds with low water content, Lactuca sativa, Eruca vesicaria, and Carum carvi, that are good examples of dry food products, and are associated with decomposition of specific classes of food products. For each seed type, the figure shows the emission of a group of volatile molecules, under different humidity conditions in the package: humid, dry and very dry.

FIGS. 6B-6D illustrate the same of the each seed type under different humidity conditions, respectively.

FIG. 7 shows the dynamics/profile of the detectable decomposition pattern during a period of 8 days, for mango fruit on a refrigerated shelf. It is shown, that the number and flow rate of specific volatile molecules characterizing the composition and rate of production of characteristic volatiles change, allowing to define the physiological age of the studied fruit, as calculated from the degree of product decomposition. In the graphs, PC1 and PC2 correspond to measurements by two sensors, respectively. PCA is the principal component analysis, which is a statistical procedure. The principles of PCA are known per se and need not be specifically explained, except to note that its operation provides for revealing the internal structure of data in a way explaining the variance in the data. In this experiment, several measurements were made every day, the corresponding measurement points are identified by different shapes in the figure. The technique of the invention enables reduction of data by summarization of data with many (p) variables by a smaller set of (k) derived (synthetic, composite) variables.

Thus, the present invention provides for effective technique for determining a true quality status (e.g. freshness) of various organic products, in particular food products, and enables users (e.g. individuals, enterprises) to access the food's true freshness/quality status. The technique of the invention enables continuous monitoring of volatile organic compounds produced during the deterioration and degradation of the organic products, and translating the detected data to the true status of the products (freshness and quality status), in a manner enabling to provide recommendations to users how this status and quality can be further taken into account for proper use of the product.

1.-22. (canceled)

23. A monitoring system for use in monitoring status of organic products, the system comprising a computer system comprising a data processing utility and being a part of and connected to a computer network, wherein the data processing utility comprises:
an input interface configured and operable for receiving input data comprising a plurality of sensing signals independently received from a plurality of sensing systems via the computer network and being indicative of volatile organic compounds sensed in vicinity of organic products over sensing time produced during deterioration and degradation of the organic products;
a data analyzer comprising a product analyzer module configured and operable for carrying out the following: extracting, from the sensing signals, one or more product-related signatures, by identifying in the sensing signals data indicative of one or more parameters of predetermined volatile organic compounds being sensed over time; and identifying, from at least one of said one or more product-related signatures, product type and real time status corresponding to said at least one product-related signature, by applying model-based analysis to the extracted product-related signature using at least one selected model data comprising multi-parameter functions describing product decomposition patterns, the multi-parameter function indicative of a sensing signal as a time function of at least one parameter for each molecule from a predetermined set of molecules of the volatile organic compounds, being sensed over the sensing time; and generating data indicative of the real time status for each the one or more organic products, thereby enabling notifying a user with management data for managing use of said products.

24. The monitoring system according to claim 23, further comprising a manager utility configured and operable for analyzing the data indicative of the product type and status, and generating notification data for managing use of said product.

25. The monitoring system according to claim 23, further comprising a communication interface utility configured and operable for data communication with a user's communication device via said computer network for communicating said notification data to the user's communication device.

26. The monitoring system according to claim 23, wherein said data processing utility is configured and operable to access and manage a database for storing data about various types of products, where each product type is associated with a respective unique set of product decomposition patterns.

27. The monitoring system according to claim 26, wherein said data processing utility is configured and operable to manage said database for storing each product decomposition pattern with associated sensing data for sensing product decomposition pattern.

28. The monitoring system according to claim 27, wherein the sensing data comprises data indicative of characteristics of one or more sensing systems from which said sensing signals are originated.

29. The monitoring system according to claim 27, wherein the sensing data comprises data indicative of one or more environmental conditions to which the sensing systems, producing said sensing signals, are exposed.

30. The monitoring system according to claim 27, wherein said product analyzer module is configured and operable for identifying the sensing data in the sensing signals being received from the sensing system.

31. The monitoring system according claim 23, wherein said model-based analysis comprises a data fitting procedure between the extracted product-related signature and the selected model data.

32. The monitoring system according to claim 23, wherein said at least one parameter of the molecule comprises either one or both of a number of the molecules of a certain type and a flow rate of said molecules being sensed over the sensing time.

33. The monitoring system according to claim 23, wherein the multi-parameter function is further indicative of the sensing signal as a function of one or more environmental conditions, which may include temperature and humidity conditions, to which the sensing system is exposed during said sensing time.

34. The monitoring system according to claim 23, wherein said data analyzer comprises a learning utility configured and operable for performing a self-learning mode for updating and improving determination of the product types and statuses, said self-learning mode comprising analyzing the product-related signatures in the independently received sensing signals relating to the same product types, and optimizing database for storing model data including various models describing product decomposition patterns characterizing various products.

35. The monitoring system according to claim 23, wherein said data analyzer further comprises a verification module configured and operable for verifying the product type by analyzing the product-related signature extracted from the sensing signal over one or more other product-related signatures in the received sensing signals.

36. The monitoring system according to claim 23, wherein said data processor is configured and operable to manage the database for storing said data about various types of products by creating and storing in said database reference data comprising measured sensing signals from a plurality of test samples of known product types as functions of time and one or more environmental conditions to which a sensing system is exposed during collection of said measured sensing signals, for various types of the sensing systems and sensing modes.

37. The monitoring system according to claim 23, wherein said data processing utility is configured and operable to access and manage a storage system being a part of and connectable to a communication network, the storage system comprising a database comprising data indicative of sensing signals from a plurality of samples of known organic product types and various freshness and/or quality statuses for each product type, each sensing signal being indicative of a multi-parameter function describing one or more parameters of each molecule from a predetermined set of molecules of predetermined volatile organic compounds produced during deterioration and degradation of known organic product type over time and one or more environmental conditions to which a sensing system is exposed during collection of said sensing signals, for various types of the sensing systems and sensing modes.

38. The monitoring system according to claim 37, wherein said database further comprises, for each product type and status, data indicative of whether and how said product with said status can be used.

39. The monitoring system according to claim 37, comprising a database comprising notification data to be provided to users of multiple organic products, said notification data comprising, for each of the multiple organic products and each of its different freshness statuses, data indicative of whether and how said organic product with the specific freshness/quality/safety status can be used.

40. A method of creating a database for use in evaluating a product status, the method being performed by a computer system comprising a processor and a non-transitory computer readable memory and being a part of and connected to a computer network, the method comprising:
a. independently receiving and storing, in said non-transitory computer readable memory, input data comprising a plurality of measured signals from multiple sensing systems via the computer network, said plurality of the measured signals comprising sensing signals measured by one or more of the sensing systems from different products of the known type and different statuses for each of said products over different sensing time intervals and different environmental conditions of said one or more of the sensing systems during the sensing times, the sensing signals being indicative of volatile organic compounds sensed in vicinity of organic products over sensing time produced during deterioration and degradation of the organic products; and
b. analyzing the input data to assign, to each type of the known product, a set of the sensing signals corresponding to product-related signature, indicative of multi-parameter functions describing sensing signal as a time function of at least one parameter for each molecule from a predetermined set of molecules of predetermined volatile organic compounds sensed over the sensing time and corresponding to product decomposition profiles over the sensing time, and the environmental conditions and sensing modes;
c. creating the database in which the product decomposition profiles are stored together with the corresponding assigned product types; and
enabling access to said database via the computer network.

41. A personal communication device configured to be a part of and connected to a communication network, the device comprising a non-transitory computer readable memory storing an application program interface comprising a manager utility configured and operable for data communication with the monitoring system of claim 23, said manger utility being configured to be responsive to input data indicative of the product-related data comprising type and freshness status of a certain organic product, for analyzing said product-related data, and generating output data comprising notification data describing whether and how said product with said status can be used.

42. A sensing system configured and operable for data communication with the monitoring system of claim 41 via communication network, the sensing system comprising: a sensing unit comprising one or more sensors configured and operable in predetermined one or more sensing modes for continuously detecting various molecules, and generating sensing data comprising data indicative of detected molecules of predetermined volatile organic compounds in a vicinity of one or more organic products over sensing time and data indicative of the sensing mode used for the detection of said molecules over the sensing time; and a communication utility for wireless communication of the sensing data to a remote monitoring system.
